(19) United States
(12) Patent Application Publication
(10) Pub. No.: US 2026/0109954 A1
(43) Pub. Date: Apr. 23, 2026

(54) Systems and Methods to Protect Biological Cells

(71) Applicant: Membrane Protective Technologies, Inc., Fort Collins, CO (US)

(72) Inventors: Lisa A. Herickhoff, Fort Collins, CO (US); Myles Shepherd, Nunn, CO (US)

(21) Appl. No.: 19/424,078

(22) Filed: Dec. 17, 2025

Related U.S. Application Data
(63) Continuation-in-part of application No. 16/766,254.
(60) Provisional application No. 62/589,422, filed on Nov. 21, 2017.
(60) Provisional application No. 62/594,394, filed on Dec. 4, 2017.
(60) Provisional application No. 63/735,020, filed on Dec. 17, 2024.

(51) Int. Cl. C12N 5/00

(52) U.S. Cl. CPC C12N 5/525

(57) ABSTRACT

Embodiments of the present invention may provide protecting, preserving, and even repairing biological cells during in vitro processing. A uniform environment such as a protective layer with exogeneous lipids may be formed around biological cells which may interact with the plasma membranes of the cells.

### PRORITY CLAIM

This application is continuation in part of U.S. application Ser. No. 16/766,254 filed May 21, 2020 which is the National Phase of international PCT application PCT/US2018/062269 filed Nov. 21, 2018 which claims priority to and the benefit of U.S. Provisional Application No. 62/589,422 filed Nov. 21, 2017 and U.S. Provisional Application No. 62/594,394 filed Dec. 4, 2017. This application also claims priority to and the benefit of U.S. Provisional Application No. 63/735,020 filed Dec. 17, 2024. Each application listed is hereby incorporated by reference herein.

### TECHNICAL FIELD

The present invention may relate to approaches in treatment of biological cells which may mitigate damages associated with environmental exposure, movement, and storage that can occur right after harvesting cells and during transportation to a processing facility and perhaps through the steps of preservation and later use or analysis. Treatment of the cells may be provided perhaps by the addition of exogeneous lipids to protect, preserve, and even restore biological cells.

### BACKGROUND OF THE INVENTION

In vitro processing of biological cells or tissues may include removal of cells from their native, perhaps in vivo, environment, transporting such cells, perhaps preserved in some manner, then utilized and/or analyzed at some future time. Specifically, there may be five steps in the processing of biological cells. A first step may be a harvesting step, then a transportation step, then a cooling or even a cryopreservation step, then a thawing or even a warming step, and finally using the cells such as implantation, fertilization, in vivo use, in vitro use, or the like. Additionally, there may be an analysis step where quality of the cells may be tested for potential use, partially used and again cryopreserved, or perhaps to ascertain the potential end result of cell or tissue use.

In each of these steps it may be important, and perhaps even vital, to optimize the environment such that the cells (e.g., tissues, organs, or the like) are able to perform at a maximum when they are later utilized. It is well understood that it may be necessary to transport cells prior processing them, such as with cryopreservation. Cells may be collected at one location but processed and even frozen at another location. As but one non-limiting example, one can consider cryopreservation of stem cells that might be collected at a hospital location where a patient may be located, then the cells may be shipped to a second location such as a laboratory having cryopreservation expertise, then perhaps later utilized for transplant into a recipient at a third location. In another non-limiting example, sperm cells may be collected at a first location of the male animal and may need to be transported to a second location for a laboratory to process the cells for cryopreservation. Therefore, sperm cells may require transportation for up to about 24 hours before processing for cryopreservation. In addition, cells or tissues collected for medical/diagnostic analysis may require transportation to a facility where they are being analyzed or utilized. As yet another example, consider organ transplants. Often patients may be too ill to travel to the location of the organ harvest therefore said organ must be transported. If such cells (e.g., tissues) cannot undergo immediate cryopreservation, the transportation process may cause detrimental changes that could change the outcome of some diagnostic analysis, or ultimate use such as in cryopreservation of gametes. Cells may need to be surrounded by compounds that may help the molecular components of the cells survive the rigors of processing.

Cells may be stored at a cooled temperature during transportation, or prior to cryopreservation which may include storage at about 4° C., about 17° C. or even at an ambient temperature. In each of these cases, the cells (e.g., tissues or organs) may continue to respire and even actively metabolize. Doing so may result in the production of oxidants, other metabolites, and even metabolic biproducts that may immediately or even ultimately harm cells. In fact, for each about 10° C. decrease in temperature, most enzymes may show about 1.5 to about 2-fold decrease in metabolic activity. Cooling may also cause a lipid phase transition which can have cumulative damage to membranous structures, acidosis, mechanical trauma, free radical production, contracture, or even apoptosis, or the like.

Moreover, it may be understood that cryopreservation may be generally a good method for preserving cells such as gametes, germ cells, unique cell lines, stem cells, bacterial, fungal, algal cells and the like. Unfortunately, cryopreservation can also negatively affect the integrity of the cell perhaps by causing changes to the lipid bilayer and perhaps even the proteins within the lipid bilayer. It may cause changes to the polarization status of the cells or perhaps even to ion channels within the cells. Oxidative damage, exacerbated by the membrane damage, may cause damage to the DNA and organelles as well. Similarly, aggregated cellular damage as described can also result in damage to tissues or organs in vitro. Such changes can be fatal to the cell or reduce the cells' potential. One or more such phase transitions may occur when cells are cryopreserved for storage perhaps in liquid nitrogen, where several lipid phase transitions occur during such process.

Cryopreservation, or perhaps cooling of cells involved in reproduction are of importance during the use of assisted reproductive technologies. These technologies are widely used in livestock. For example, nearly 90% of dairy cows are artificially inseminated. Similarly, nearly all sows (female pigs) are artificially inseminated. A large percentage of show cattle, goats, and sheep are propagated by artificial insemination and embryo transfer. In many of these cases, the cells (perhaps sperm or oocytes), or tissues (perhaps embryos) are cooled from body temperature, frozen in straws, in droplets or similar devices using perhaps nitrogen then thawed to body temperature and implanted in the body of a recipient. As another example, cryopreserved sperm cells may be sorted into X-and Y-bearing cells pre-freeze or post-thaw, then utilized for an in vitro fertilization process. In this case, the damage to the sperm cells induced by cryopreservation may impact the quality of the embryo ultimately produced.

When cryopreserving reproductive cells, sperm, oocytes or embryos, they are in a medium or various media that must accommodate a temperature transition from 37° C. to perhaps 4° C., to perhaps −20° C., or −196° C., to perhaps even as low as −230° C. After storage, the cells may be thawed and must again transition to about 4° C., 22° C. or 37° C. depending on species and end use. Such a temperature transition can be detrimental to cellular compounds and molecular mechanisms. Damage may include damage to the molecular structure and integrity of the plasma membrane, changes in membrane fluidity, increased lipid saturation, changes in lipid aggregation, functional changes such as acrosomal membrane integrity changes, mitochondrial function damage, as well as follow-on DNA damage by moieties now able to pass through the membranes and into the nucleus.

The lipid composition of membranes may have a direct effect on the ability to cryopreserve a cell. In the past, attempts have been made with varying degrees of success to add cholesterol and similar fluidity inducing fatty acids to sperm cells. In addition, lipids have been added to culture medium when cells are growing.

It is generally understood that biomembranes homeostatically adjust their lipid composition to maintain the appropriate intrinsic curvatures in each leaflet of the bilayer. However, once cells are in vitro, they are no longer able to adjust. Cholesterol is a normal part of lipid bilayers of plasma membranes and as such has been used to artificially change the stability of a lipid bilayer. However, cholesterol may not be able to physiologically correct the biomembrane functioning of the cell. Because of its generally flat structure, it may behave amphipathically and serves as a type of ‘glue’ for a bilayer. But, it does not appear to be randomly distributed and therefore the integration of additional, exogenous cholesterol or perhaps even the depletion of said endogenous cholesterol may not appear to provide benefits in maintaining cellular function and especially during phase transitions as may occur during cryopreservation, cooling, or other storage conditions.

It is desirable to find an appropriate type of exogeneous lipids that can be added to a biological cell to protect, preserve, and even restore the plasma membranes of biological cells during in vitro processing, cryopreservation, and the like.

### DISCLOSURE OF THE INVENTION

The present invention includes a variety of aspects which may be selected in different combinations based upon the particular application or needs to be addressed. In one basic form, embodiments of the present application provide treatment and even preservation of biological cells which may encompass processes beginning at harvesting the biological cells to shipping the cells to use of the cells. In addition, embodiments of the present invention may include methods and systems which protect biological cells such as by providing uniform environments, protective layers, and the like around each cell.

Methods to modify membrane composition have been explored as a method to protect from cryopreservation damage, primarily the use of cholesterol and PE. However, the utilization of an ancillary lipid layer monolayer, or secondary lipid structures, to support the existing cellular bilayer has not been explored. Embodiments in this application provide a method to provide and/or create an isotropic non-lamellar phase of lipids which can support the cell's bilayer and thereby stabilize its functionality, the cell's ionic potential, and physical structure. Non-bilayer lipids and non-lamellar lipid phases provide a method to protect, perhaps even enhance or beneficially modify, the functionality of in vitro lipid bilayers of protected cell, cells, tissues and perhaps even organs.

Biological membranes may naturally contain non-bilayer lipid species which do not self-assemble into bilayers. The utilization of such non-bilayer species to create a protective layer associated with a cell's native bilayer may provide a method to enhance physiological function both during in vitro use, storage or treatment and also during subsequent in vivo uses. It is desirable to protect the native membrane composition of in vitro cell, cells, tissue, organs, and the like from damage inflicted during physical or temperature transitions including cooling, freezing, storage, thawing, and perhaps even refreezing and thawing again. This protection imparts integrity to the cell such that it is able to function more like a cell that has not undergone thermal stress.

Naturally, further objects, goals and embodiments of the inventions are disclosed throughout other areas of the specification, drawings, and claims.

### BRIEF DESCRIPTION OF THE DRAWINGS

FIG. 1 provides a conceptual representation of various embodiments of the present invention.

FIG. 2 provides a conceptual representation of a biological cell in an uniform environment as may be understood in the various embodiments of the present invention.

FIG. 3 provides a conceptual representation of a biological cell in an environment as may be understood in the various embodiments of the present invention.

FIG. 4A provides a flow cytometric dot plot of sperm stained with Merocyanin 540 and YO-PRO-1 of a control group where sperm were cryopreserved with a traditional medium, then thawed and analyzed.

FIG. 4B provides a flow cytometric dot plot of sperm stained with Merocyanin 540 and YO-PRO-1 where enhanced sperm were treated additional exogeneous lipids, then cryopreserved, thawed and analyzed.

FIG. 5 provides a bar graph demonstrating the difference in plasma membrane reactivity through use of exogeneous lipids.

FIG. 6 provides a conceptual representation of processing a biological cell in accordance with various embodiments.

FIG. 7A provides a characterization of a protective layer which may be added to a cell perhaps to confer protection to a biological cell as may be understood in the various embodiments.

FIG. 7B provides a characterization of a protective layer which may be added to a cell perhaps to confer protection to a sperm cell as may be understood in the various embodiments.

FIG. 8 provides a bar graph demonstrating the difference between sperm that has been cryopreserved, then thawed with a control group medium and with an enhanced group medium as may be understood in the various embodiments.

### MODE(S) FOR CARRYING OUT THE INVENTION

As mentioned earlier, the present application includes a variety of aspects, which may be combined in different ways. The following descriptions are provided to list elements and describe some of the embodiments of the present invention. These elements are listed with initial embodiments; however, it should be understood that they may be combined in any manner and in any number to create additional embodiments. The variously described examples and preferred embodiments should not be construed to limit the present invention to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments are not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Embodiments of the present application may provide methods and systems for protecting transported biological cells with synergistic continuity. It may be a full system perhaps considering five steps in processing of biological cells which may enable the decrease in damage of cells over the course of processing and even subsequent increase in post-processing cellular health. The synergistic continuity achieved by developing a fully integrated system, rather than a pieced system, may allow increased success of the cells, tissues or organs at the final point of use. By coordinating individual steps, one may decrease the processing time of the whole system thereby limiting damage to the cells. Compatibility within the system may allow protection at each individual step from components or compounds that may later cause a decrease in effectiveness. Much of the damage within in vitro systems may be cumulative. By limiting the initial damage, the accumulation of total damage can be decreased.

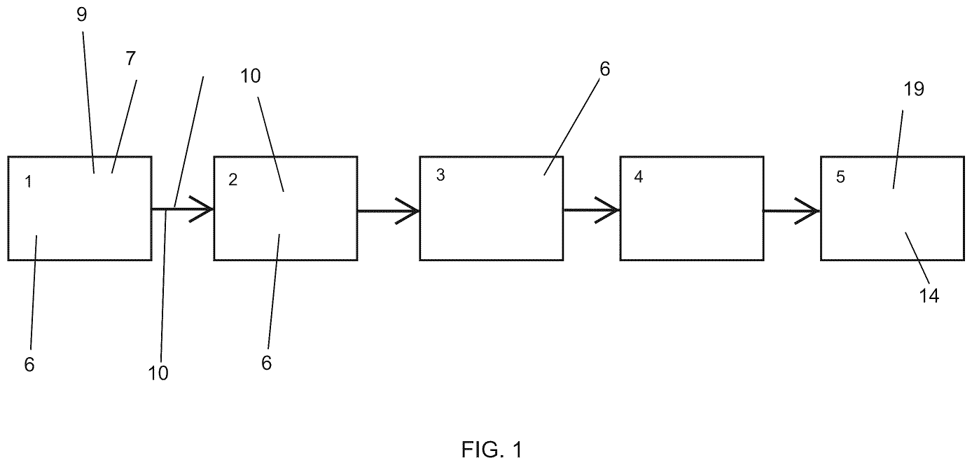

Embodiments may consider the individual steps for processing but rather than optimizing the individual steps, optimizes the entire system for items that occur within each of the steps. As may be understood from the conceptual diagram of FIG. 1, a first step in a processing system may be a harvesting step (1) where a collection of biological cells (6) may be harvested from an in vivo source (7), a second step may include transporting (2) biological cells, a third step may include hypothermic treatment (3) of said biological cells, a fourth step may include warming (4) of the biological cells, and perhaps even a fifth step of using (5) the biological cells.

Biological cells can be utilized for a variety of functions after collection, after cooling, or even after return to a physiologically active temperature (e.g., thawing to perhaps about 37° C.). Such functions may include but is not limited to, fertilization, insemination, fertility, intrauterine insemination, laparoscopic insemination, intracervical insemination development of an embryo, development of a conceptus, development of a sustainable pregnancy, transfer of an embryo from one animal to a recipient, biological purposes, centrifugation, density gradient treatment to remove excess materials such as residual diluent, cryoprotectant, and/or to concentrate cells, flow cytometric assessment or sorting for a variety of characteristics including sex sorting, in vitro fertilization, further or subsequent cryopreservation, intracytoplasmic sperm injection, and the like. Cells treated with additional exogenic lipids and that may have a protective layer around at least part the cells may be able to undergo post-thaw, or post-warming functions with more success perhaps resulting in higher fertilization rates, improved embryo quality, improved pregnancy rates, improved post-thaw functionality, the ability to be utilized for additional in vitro handling, and the like.

Embodiments may include maximizing viability of biological cells including establishing a uniform environment around each biological cell of a collection of biological cells, which can protect and even preserve the biological cells during or after hypothermic treatment, warming, and perhaps even using said biological cells. A uniform environment may include a protective layer around at least part of a biological cell.

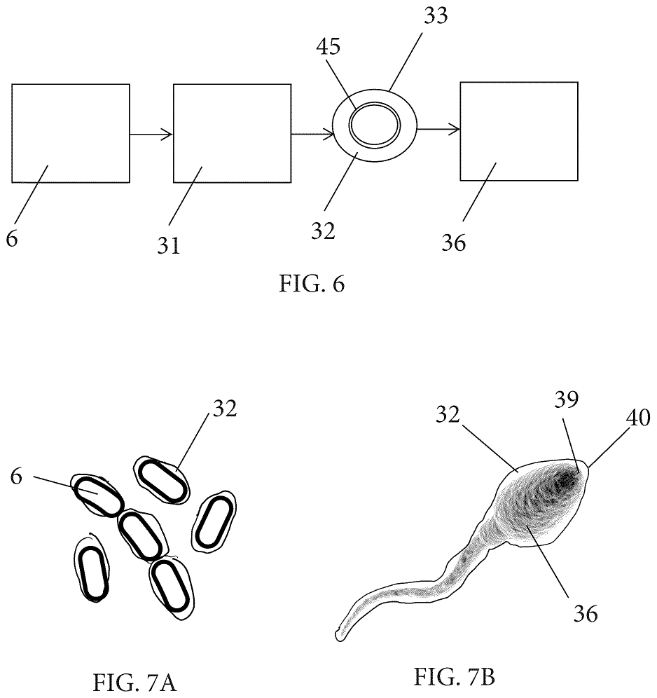

Embodiments may provide methods and systems for processing biological cells including the development and addition of a lipid layer that may be continuous over at least a portion of a critical functional area, a functional area, or perhaps an entirety of the cell. As such this embodiment may have innate functional impacts that are included as further embodiments of the present application. In embodiments and as may be understood in FIGS. 6, 7A, and 7B, a biological cell (6) may be provided, exogeneous lipids (31) may be added to the biological cell perhaps before, during, or even after in vitro processing (36) of the biological cell. Then, a protective layer (32) of the exogeneous lipids (31) may be formed around at least part of the biological cell to perhaps create a stabilized biological cell (33) which may be in vitro processed and result in substantially preserving a plasma membrane (34) of the biological cell during the in vitro processing. In some instances, a protective layer may substantially preserve a polarized state (35) of the biological cell during in vitro processing. FIG. 7A shows an example of a protective layer (32) around a biological cell (6) and FIG. 7B shows an example of a protective layer (32) around a sperm cell (36). A protective layer of the exogeneous lipids may be formed around at least part of a biological cell, around at least part of the plasma membrane of a biological cell, over at least part of a plasma membrane or cell, around most of a cell or its membrane, or even around all of a cell or its membrane. The protective layer may preserve the plasma membrane of the cell during in vitro processing which may protect the plasma membrane from damage. By substantially preserving a plasma membrane, the plasma membrane may be understood in a dictionary sense as a term that encompass an ample or considerable amount, quantity, size, etc. as well as terms that encompass largely but not wholly that which is specified. As such, there may be some damage but not an ample amount of damage.

Biological cells (6) may include, but is not limited to, reproductive cells, cells, tissues, sperm, equine sperm, bovine sperm, caprine sperm, ovine sperm, porcine sperm, fowl sperm, ovaries, oocytes, embryos, organs, stem cells, genetically modified cells, artificially derived cells, any combination thereof, or the like. A collection of biological cells (6) may be harvested from an in vivo source (7) which may include, but is not limited to, mammal, human, rodents, equine, bovine, caprine, ovine, porcine, fowl, fish, shell fish, reptile, nephropidae, poikilothermic, aquatic vertebrates, or the like.

As a non-limiting example, uses and in vitro processing of the biological cells may include insemination, implantation, culturing, research, diagnostic testing, replication, gamete preservation, genetic preservation, a first, and even a second cryopreservation, reproduction, any combination thereof, or the like.

Embodiments may include a method and formulation for protecting cells during further processing such as freezing. For example, systems may include protecting cells during concentration steps such as centrifugation or filtration, during media changes or transitions, during temperature transitions, and the like. Embodiments may be utilized to flush oocytes or even embryos and store them during transportation to a laboratory. Embodiments may also include devices appropriate to enable such utilization.

An embodiment may include the development and addition of a fluid lipid layer such as a protective layer (32) that may be continuous over a portion, a critical functional area, a functional area, or perhaps the entirety of the cell. As such this embodiment may have innate functional impacts that are included as further embodiments of the present invention.

A protective layer (32) of exogeneous lipids may be a fluid lipid layer that can absorb or otherwise subvert damage to a cell's plasma membrane and its associated lipid bilayer perhaps induced by in vitro processing and even thermotransitions. This may provide an enhanced ability of a cell to maintain in vivo lipid domains during in vitro and/or temperature transitions and thus may maintain the biophysical property of the membrane. Specific local structures in the lipid bilayer may impact functionality so preserving the membrane phases may be important to maintain specific functionality.

An embodiment may include the ability create an enhanced transition, and tolerance from, the L phase (liquid crystalline) at elevated (or physiological temperatures) to Hn or other similar phases at different temperatures. An embodiment may include the ability to maintain cellular lipid raft domains that enhance functionality of the cells as the cell transitions through temperatures and in vitro handling and manipulations. An embodiment may include the maintenance of either liquid crystalline bilayer phases, non-liquid crystalline and non-bilayer solid, or gel phases favored by low temperature and long chain fatty acids.

An embodiment may include the lipid composition such that a non-lamella, monolayer, and even a non-bilayer may be formed as a protective layer for a cell. An embodiment may include the appropriate balance of lipids to modulate protein energetics. Protein energetics can define the interaction of proteins and lipid bilayers to enable crucial cellular processes and functionality. Such modulation may enable the appropriate embedding of proteins and membrane-protein interactions. By maintaining such protein positioning through temperature transitions, the natural physiological function and structure may be maintained. An embodiment may maintain the lateral heterogeneity of the protein compositions of an in vivo membrane within an in vitro system. A further embodiment may assist in retaining protein-membrane interactions and protein functions.

A plasma membrane (45) including a healthy plasma membrane may be formed of endogenic lipids, lipid bilayer, proteins, lipid rafts, carbohydrates, and associated complexes. A protective layer of exogenic lipids may provide protection, preservation, and even restoration to a plasm membrane including maintaining lipid rafts and the like.

An embodiment may include an added lipid composition such that processes similar to the Dynamic Exchange Model may be achieved. This may be the coexistence of bilayer and non-bilayer phases in a dynamic equilibrium as previously described for thylakoid membranes in plants. Further, the dynamic exchange model refers to the concept that lipids and membrane-associated proteins are not fixed in place within a single leaflet, but instead undergo continuous, thermally driven movement and exchange between structures, domains, or even between leaflets. It may explain how membranes maintain fluidity, asymmetry, domain formation, and responsiveness while still allowing local structure (such as lipid rafts or protein clusters). The dynamic exchange model describes membranes as constantly shifting, self-organizing structures where lipids and proteins: move laterally, enter and leave microdomains, can exchange between leaflets and perhaps are reshaped by trafficking and mechanical forces. This dynamism may allow cells to maintain stable structure while being highly responsive.

This model further posits the self-assembly and structural dynamic that depend on the fusion of membranes and intermembrane exchange of lipids and may portray the bilayer phases and describe the lipid interactions within a bilayer and includes protein networks and pathways along the surfaces as well as impermeability of the bilayer. Embodiments may include maintaining and even repairing the functional plasticity of a lipid layer determined by lipid phase behavior. This model further posits that non-bilayer phases play a role in insertion and assembly of proteins within lipids within a bilayer. Embodiments herein can leverage such model to be utilized in the protection of cells destine for cryopreservation and the like.

In an embodiment, such exchanges may be mediated by the concentration variables which are not independent. Lipid concentrations in both the lipid bilayer and the non-bilayer phases may further affect lipid exchange. An exchange direction may be driven by the molar percentages within the two membranes and an exchange may create an equilibrium between the two membranes. Such directional flow may also be coupled to membrane configuration and such an exchange may drive additional membrane stability which aids the cell in thermos-transitions.

In an embodiment, lipids may be exchanged between the phases such that one phase is stabilized. Such lipid transfer may be mediated by lipid transfer proteins to enable more rapid transport than vesicular transport. They may act as a shuttle. Such lipids may be exogenously added or may be endogenous to the cell or surrounding fluids such as seminal plasma.

As such, a protective layer of exogeneous lipids may interact with a plasma membrane of a biological cell and may protect the biological cell from damage from in vitro processing such as to provide a stabilized biological cell or even a membrane-stabilized biological cell. This protective layer may even substantially preserve a polarized state, substantially preserve an electrical or ionic membrane potential of a biological cell, substantially maintaining a zinc-ion efflux ability of a sperm cell, or even substantially maintaining a zinc signature state of a sperm cell.

Substantially may be understood in a dictionary sense as a term that encompass an ample or considerable amount, quantity, size, etc. as well as terms that encompass largely but not wholly that which is specified. In embodiments, a protective layer may be used to preserve a polarized state of a cell up until it may no longer needs to be preserved such as when a cell is at an appropriate place in uterine tract, when a biological cell is used for in vitro fertilization, and even when a biological cell is used for in vivo fertilization.

An anticipated cell damage limiting regimen (19) may include an expectation of what kind of cell damage may occur to a specific type of biological cells perhaps for a specific type of use and trying to limit such damage by modifying the process. Non-limiting examples of an anticipated cell damage limiting regimen (19) may include a reduction in cell damage perhaps caused from an aspect such as biological contamination, chemical contamination, contamination caused by invasive species, chemical residues, detergents, disinfectant residues, solvent compounds, organic compounds, photo activation, photo modification, improper handling, bacteria, fungi, mycoplasma, virus, any combination thereof, or the like.

The present invention, in embodiments, may provide media or medium (10) perhaps applicable for an anticipated cell damage limiting regimen and even a predetermined use. A media (whether holding, cryopreservation, thawing, re-freezing and the like) may include components which may be tailored to limit cell damage for a system and may even be tailored for a predetermined use. A media (10) may include, but is not limited to, natural ingredients, non-animal derived components,, membrane stabilizing compounds, membrane protective compounds, lipids, phytochemicals, secondary metabolites of plants, plant extract, any combination thereof, or the like.

Embodiments of the present invention could address a means of adding media or media components including lipids, perhaps at a metered rate during shipment such that the protection conferred by the moieties may not be depleted over the time of the shipment. This could include time release (e.g. time released compounds in a holding media), increased quantities (e.g., adding enough holding media to last throughout a transportation step), metered or even drip addition (e.g., adding additional media during a transportation step), and the like to keep biological activity perhaps at an optimum level. Moreover, embodiments of the present invention may include, within the system, the addition of various compounds at all points of the processes. In some embodiments, the invention may limit the changes of media within the processing perhaps by adding various compounds at the beginning to increase effectiveness throughout the entire process.

In some embodiments, the present invention may include methods and systems that may leverage a cooling & shipping methodology and may extend it to a methodology for cryopreservation, and/or preparation for cryopreservation.

Embodiments of the present invention may provide a composition of free fatty acids, phospholipids, glycosyldiacylglycerols, bilayer and non-bilayer lipids, perhaps in the range of about 0.5% to about 10% v/v to provide stabilization to membranes to protect the cells from exposure to oxidative compounds, to prevent cell disruption, cell rupture and expulsion of cytoplasmic compounds that may be injurious to adjacent, intact cells, individually and any combination thereof.

Other embodiments of the present invention may provide a method or device to limit exposure of the cells and surrounding solutions to damaging moieties.

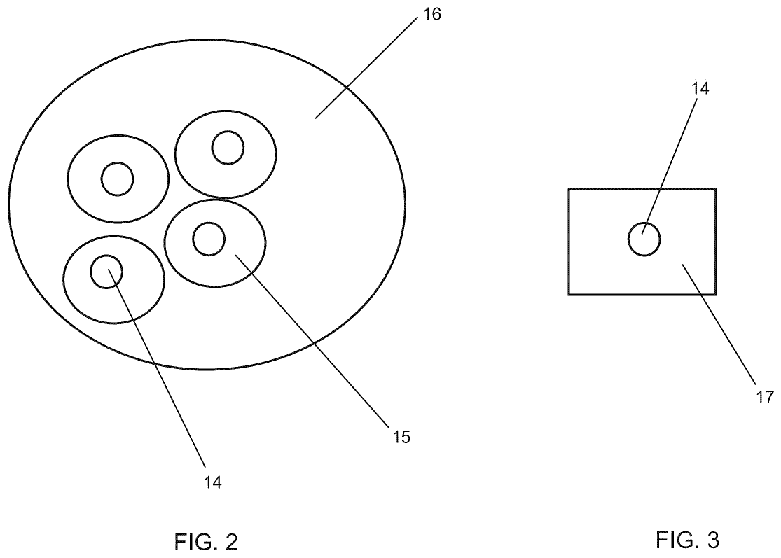

As may be understood from the conceptual representation provided in FIG. 2, a biological cell (14) may be encapsulated or even surrounded by a uniform environment (15) which may then be surrounded by a media (16). FIG. 3 shows an example of a conceptual representation of a cage-like structure (17) around a biological cell (14). A cage-like structure may be a protective layer using exogeneous lipids. Lipids can form a cubic phase, even small micelles, creating perhaps a cage-like structure perhaps as a three-dimensional complex. These structures may be defined as non-bilayer lipid phases or bilayer phases. In addition to perhaps bilayer phases, they may contain Hn phase, L-phases and other isotropic phases.

Embodiments of the present invention may relate to the ability to add compounds which may compensate for the endogenous (or native) lipids which may be inhibiting or conversely enabling membrane fluidity. Such lipids may be external to the cell, may be attached to the cell, or may even be incorporated into the cell. A choice of interaction of the lipids with the membrane may be dependent on the cell type, the ultimate goal for use of the cell, the type of storage media, the type of desiccation and/or cryopreservation method utilized, or any combination thereof, and the like. Embodiments may relate to a method of creating and maintaining such a customized environment perhaps by encapsulating at least part of the cells.

Some embodiments of the present invention may include any commonly identified membrane lipids, may be free fatty acids, may contain phosphoglycerides, galactolipids, glycosylolacylglycerols, glycogylcerolipids, non-bilayer lipids, or sphingolipids, and may also contain membrane proteins, salts, agarose, or other materials. Such materials may interact with the phospholipid head group to form a cage-like structure (17) that may stabilize the lipids and/or lipid rafts in the cell membranes. Exogeneous lipids may include galactolipids, phosphatidyl glycerol, palmitoleic acid, phospholipids, plant derived phospholipids, animal derived phospholipids, any permutation and combination thereof, and the like. Galactolipids may include monogalactosyldiacylglycerol (MGDG), digalactosyldiacylglycerol (DGDG), sulfoquinovosyldiacylglycerol (SQDG), any permutation and combination thereof, and the like.

Other lipids may have a different effect on the membrane shape and functionality. Adding phosphatidylethanolamine (PE) and/or phosphatidylserine (PS) for example may cause the organization of different structures within a cellular lipid layer. As but one non-limiting example, PE, and PS, may form a conical-type lipid structure and may also create inverted micelles. PC (phosphatidylcholine), PG (phosphatidylglycerol), PI (phosphatidylinositol) may form a lamellar phase with no curvature, and lysolipid may form an H1 monolayer (tubule or spherical micelle). Each of these lipid polymorphisms may cause either detrimental or perhaps even beneficial changes within the lipid bilayer of an in vitro cell.

Different polar head groups affect shape of the polar lipid as they aggregate. The addition of other free lipids such as PE, phosphatidic acid (PA), or lysophosphatidylcholine (Lyso-PC) can cause the formation of non-bilayer phases; they can form a cylinder, cone or inverted cone shaped. A grouping of one or more of these lipid may result in the formation of a different membrane shape, may cause lipid rafts, or may cause changes in the permeability of such lipid bilayers if they are integrated. While considering the potential for lipids to integrate into a membrane of an in vitro cell it must be acknowledged that such changes may be affected by other environmental influences within the cell's in vitro medium. Changes in temperature, hydration, pH and NaCl concentrations may cause changes in lipid organization that can supersede the natural impacts of the polar head groups. Moreover, it may be generally understood that in vivo lipid bilayers contain lipid microdomains where specific types of lipids aggregate which further exacerbate the challenges of utilizing such to create functional changes.

The success of the integration and use of different polar head groups during cryopreservation may not be certain. In fact in some cases may have caused functionally detrimental events such as capacitation. Capacitation as the final maturation step for sperm, causes irreversible changes in the membrane permeability to enable a sperm cell to fertilize an oocyte. As previously mentioned, such changes may be detrimental to membrane functionality and affect other cellular, organelle or organ-level abilities. Such changes may be further exacerbated when one considers temperature changes that a cell may undergo during long-term storage such as cryopreservation and subsequent thawing.

The application may provide forming a protective layer that can mimic (40) an intrinsic curvature (39) of a cell's plasma membrane as may be understood in FIG. 7B. A protective layer may be a isotropic, non-lamellar protective layer. Mimicking an intrinsic curvature of a plasma membrane may be helpful in protecting and even preserving a biological cell during processing. Such protective layer may inhibit a substantial change of the intrinsically functioning plasma membrane during in vitro processing.

In some embodiments, a protective layer of exogeneous lipids may repair a damaged plasma membrane of a biological cell. A damaged plasma membrane may result from in vitro processing, thermal transitioning, may have flipped phospholipids, oxidation of membrane lipids, a change in the original cell curvature, or the like. Exogeneous lipids of a protective layer may: (1) interact with the damaged membrane to repair lipid rafts; (2) integrate the exogenous lipids with endogenic lipids of the damaged plasma membrane; (3) restore at least part of a lipid bilayer of the damaged plasma membrane to a non-damaged intrinsically functioning lipid bilayer; (4) restore at least part of a lipid bilayer of said damaged plasma membrane to a non-damaged intrinsic curvature of a lipid bilayer; (5) form a isotropic, non-lamellar protective layer over the damaged plasma membrane; and the like. The exogeneous lipids may be added to the biological cell at any time, including, but not limited to, during biological cell collection; immediately after biological cell collection; before cooling of said biological cell; after cooling of said biological cell; before cryopreservation of said biological cell; after cryopreservation of said biological cell; before in vitro fertilization event of said biological cell; before in vivo fertilization event of said biological cell; before cell sexing of said biological cell; after cell sexing of said biological cell; before cell sorting of said biological cell; after cell sorting of said biological cell; before centrifugation of said biological cell; after centrifugation of said biological cell; before in vitro treatment of said biological cell; before a second cryopreservation of said biological cell; and after a second cryopreservation of said biological cell. Further, a protective layer may be used to protect a damaged plasma membrane during processing, thermal transitions, cryopreservation, a second or subsequent cryopreservation, and the like.

In embodiments the formulation of the exogeneous lipids may be optimized based on different factors including but not limited to, the conditions of the biological cell, where the conditions may be pH, osmotic pressure, temperature, or the like; an appropriate type of exogenous lipids to return an ionic state of the biological cell to a healthy ionic state; an appropriate type of exogenous lipids to add based on the type of in vitro processing done to said biological cells; to preserve an ionic state of the biological cell; based a composition of a plasma membrane.

Embodiments of the present invention may contain an unusual distribution of lipids such as linolenic acid (18:3) at approximately 40%, linoleic (18:2) at about 15% and palmitic at about 20%. Lipids may be commonly found in the species of the plant or animal or may be lipids not found within the family, genus or species. Lipids may also be from a different phylogenetic kingdom, such as plants with animal cells and the converse. In some embodiments, a blend of lipids, free fatty acids, phospholipids, glycosyldiaclglycerol lipids, and the like may be added to biological cells which may be optimally beneficial to an individual cell type and a cell derivation. An embodiment may include the combinations of galactolipids, mono- and di-galactosylidiacylglyerol (MGDG and DGDG, respectively), and phospholipids, phosphatidyglycerol, sulfoquinovosyldiacylglycerol, 3E-Hexadecenoic acid, and even perhaps palmitoleic acid.

In embodiments, such exchanges may be mediated by the concentration variables which are not independent. Lipid concentrations in both the lipid bilayer and the non-bilayer phases may further affect lipid exchange. An exchange direction may be driven by the molar percentages within the two membranes and an exchange may create an equilibrium between the two membranes. Such directional flow may also be coupled to membrane configuration. Such an exchange may drive additional membrane stability which aids the cell in thermos-transitions.

An embodiment may utilize phosphatidylinositol or phosphinositides to act as a lipid ligand, or perhaps to create a lipid gradient.

An embodiment may include up to about 1% of total of each of the fatty acids palmitoleic acid, about 1% total galactolipids as DGDG or MGDG, about 1% phosphatidylglycerol and perhaps up to about 1% of each of the other phospholipids. In other embodiments, up to about 3% exogenous lipids v/v may be added to a biological cell or even up to about 3% total exogenous lipids in a solution may be added. A solution may be an extender. Of course, any amount of exogeneous lipids may be added as needed.

As mentioned, the application may provide adding exogeneous lipids to a biological cell before, during, or after said in vitro processing. This may include but is not limited to adding exogeneous lipids to a biological cell: during or after collection of a biological cell; before, during, or after thermal processing of a biological cell; before, during, or after cooling a biological cell; before, during, or after cryopreservation of a biological cell; before, during, or after cryopreservation of a biological cell and before use of a biological cell with in vitro fertilization; before, during, or after cryopreservation of a biological cell and before use of the biological cell with in vivo fertilization; before, during, or after cryopreservation of a biological cell and before use of the biological cell with intrauterine insemination; before, during, or after cryopreservation of a biological cell and before sorting of the biological cell; before, during, or after cryopreservation of a biological cell and before sexing of a biological cell; before, during, or after cryopreservation of a biological cell and before centrifugation of a biological cell; before, during, or after a second cryopreservation of a biological cell; any combination or permutation; and the like.

The present invention, in embodiments, may include methods to encapsulate the lipids such that they can be integrated into the membrane. Such encapsulation may include liposomes, or similar methods to enable the separation, the addition and the incorporation of the lipids as the invention may dictate.

An embodiment may include a different balance of phospholipids to enable the adaptation to pH changes that may occur in both the in vitro state as well as the subsequent in vivo state when the cell is utilized for its intended purpose.

An embodiment may include the appropriate physio-chemical environment (pH and ionic strength) of the media used with said cell, cells, tissues or organs, to maintain the appropriate lipid mono- and bi-layer interactions.

An embodiment may additionally include non-bilayer lipids in the self-assembly of a protective system. Such may also include free lipids which assemble into structures loosely associated with the cell membranes.

Embodiments may include that decreasing permeability to foreign compounds such as oxidants and other potentially harmful substances within the environment of the in vitro cell, cells, tissues or organs. Such embodiment may also include permeability to thermo-transitioning moieties including ice crystals.

An embodiment of a nonbilayer lipid phase may significantly contribute to the structural dynamics of a fully function membrane. An embodiment may enable the appropriate biological transition from an in vitro functionality to an in vivo functionality when a treated cell is used for its intended function. As but one non-limiting example, a sperm cell held in vitro may be protected from damaging events. When the sperm cell is deposited into the cervix or uterus, having a different physio-chemical environment, the monolayer may disassociate from the cell, enabling it to be vulnerable to oxidants that induce capacitation. Such transition is necessitated for fertilization.

Exogeneous lipids may include a blend of lipids, and lipids with appropriate length acyl chains to affect both kinetic and thermodynamic properties of the lipid phases required for the disclosed invention to function correctly during the temperature transitions or functional needs. Such functional needs may include fusions of said lipids, or lipid-structures. Lipids of different phases may be included within the invention within a complex of lipids. As but one non-limiting example, membrane lipids might include isotropic phase lipids within a lipid bilayer. Some embodiments may include the creation of a ‘bubble’ or ‘blanket’ of beneficial compounds surrounding the cell which may limit changes in the membrane composition of the cell itself.

Embodiments provide a specialized method to optimize or standardize the environment surrounding the cells during treatment or exposure to the invention. Such a method may include a method to encapsulate a small grouping of the cells into a limited uniform environment. Such methods may include a microfluidic type system to create a microenvironment. Encapsulating a biological cell may be accomplished by micellular structure; a lipid layer, a lipid monolayer, a lipid bilayer, or the like. The microenvironment may include antioxidants, plant lipids, animal-derived lipids, synthetic lipids, and/or any number of a variety of moieties which may be known to be beneficial. The microenvironment may include any of the aforementioned attributes such that the continuity of the system and indeed of the cellular or tissue environment is maintained over the entire course of processing. While said system may not have been previously used to encapsulate cells such as sperm for use in artificial insemination, this system may provide one method for development of an appropriate microenvironment to encapsulate a small number of cells.

Embodiments of the present invention may be applicable to a wide variety of commonly utilized media, buffer or extenders for cryopreservation, shipping, thawing, tissue preservation cells, tissues or organs.

Lipids may include lipids, free fatty acids, phospholipids, proteins, glycoproteins, glycosyl diacylglycerols, diglyceride, monoglyceride, lipoproteins, and other compound containing lipids and the like as might be described above. Additional compounds may include include sugars, salts, proteins, compound molecules, phytochemicals, secondary metabolites of plants, and similar moieties that might all function in a similar, or substantially similar, manner, or may aid the lipids in acting and reacting in a predictable manner. Further the selection of fatty acids may be such that they are optimized for the temperature and phase transitions that the cell, cells, tissues or organs may undergo in an in vitro or in vivo state.

An embodiment may be the stabilization, or perhaps even the beneficial reorganization of membrane components such as perhaps biochemical markers, proteins, ion pumps, cellular receptors, and the like by application of the disclosed invention.

An embodiment may be the protection of cellular membranes that have been cooled to 17° C., 4° C., or have been frozen to 0° C., −20°C., −196°C. (LN2 vapor), −210° C. (LN2 liquid). Such temperature transition may include vitrification, slow freeze, freeze-drying, subzero nonfreezing storage, preservation in a dry state and similar techniques as may be necessary to maintain the cell in a preserved state for an extended period of time.

An embodiment may include the pre-freeze or post-cooling use of said invention treated cells for in vitro fertilization, intracytoplasmic sperm injection, sorting into X-and Y-bearing chromosome cells, allogenic cell therapies, cloning, and the like. Pre-freeze cells may be rendered sufficiently robust to undergo additional treatments such as centrifugation, high-yield cell harvest, additional cryopreservation, and the like. Such pre-freeze or post-cooling treatments may include manual or automated cellular systems.

An embodiment of the invention may include the post-thaw use of said treated cells for in vitro fertilization, intracytoplasmic sperm injection, sorting into X-and Y-bearing chromosome cells, allogenic cell therapies, cloning, and the like. Post-thaw cells may be rendered sufficiently robust to undergo additional treatments such as centrifugation, high-yield cell harvest, additional cryopreservation, and the like. Such post-thaw or post-cooling treatments may include manual or automated cellular systems.

An embodiment may enable a second freeze of the tissue or cells through treatment to repair damage from the first cryopreservation procedure. Said cells or tissues may not have been treated with said invention prior to the first freeze, or may have been exposed to it for both freeze cycles.

Another embodiment may include maintaining, or improving critical parameters such as viability, motility, DNA quality, therapy-specific critical quality attributes, functionality, membrane quality, acrosome quality, membrane protein maintenance, membrane orientation and fluidity maintenance, protein association and the like.

Embodiments of the present invention may provide a method and supplement to assist in the transition between room temperature, cooling, freezing, and perhaps even implantation, insemination or further culturing, fertilization, cryopreservation, or other uses.

An embodiment may include use with a variety of cells, either those collected from an in vivo source, those cultured as a single cell, from a primary cell culture, or derived from a cryopreserved source then cultured. Cells may also include those derived from a variety of in vivo sources then combined to create a new compendium of cells. As but one non-limiting example, sources may include sperm cells, oocytes, embryos, blastocysts, embryo-like structures, expanded potential stem cells, stem cells, trophectoderm cells, cells derived from, or biopsied from mammalian organs or tissues, adipose-tissue derived stem cells, amniotic fluid, bone marrow, mammalian cells, synovium, dental pulp, embryonic stem cells microorganisms, umbilical cord, hepatocytes, red blood cells, teeth, and pancreatic islets.

An embodiment may include storage for a short, or long period of time depending on the temperature, cell type, and final use.

Embodiments of the invention may include the ability to be packaged or encapsulated in such a way that enables the further use for perhaps insemination, re-cryopreservation and the like. Encapsulation methods may include, but are not limited to, a 0.25 cc straw, a 0.5 cc straw, an ampule, a Eppendorf tube, a test tube, an insemination rod, a cannula, an insemination device, a drop of nitrogen, and the like.

### EXAMPLE 1

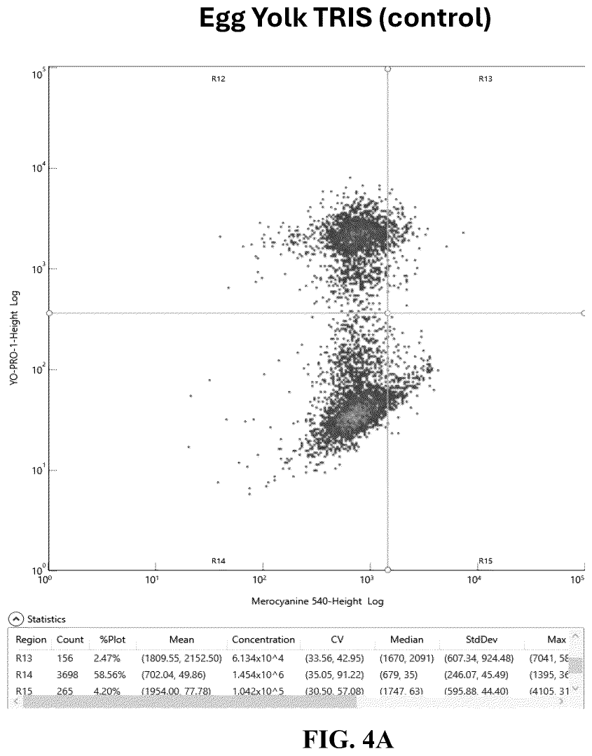

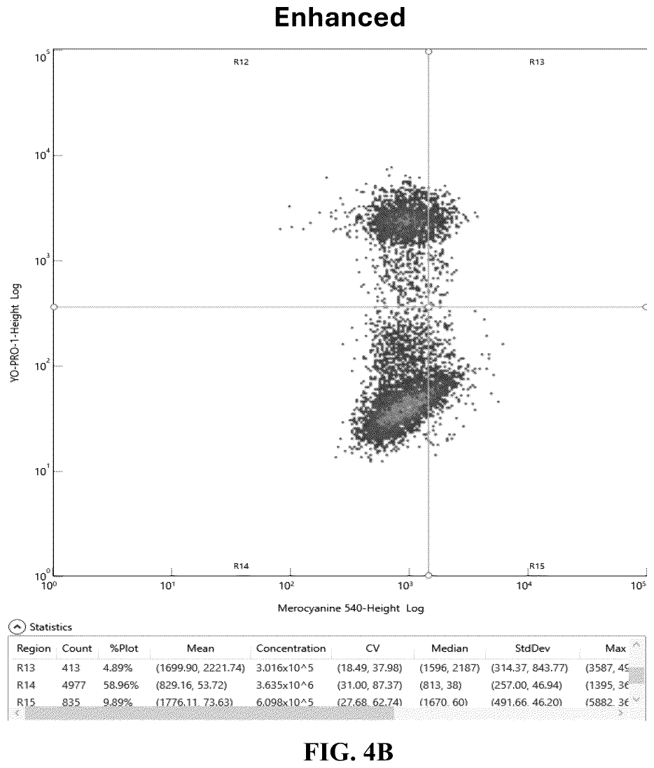

As a demonstration of the impact of exogeneous lipids from an exogenous source and the protective nature of a lipid protective layer on cryopreserved cells, a split ejaculate study was executed. In a split-ejaculate study, raw semen from 12 bulls was split equally into one of two extenders, one a traditional egg yolk extender and the other egg yolk extender plus 1% of totally fatty acids as palmitoleic acid, 0.05% total galactolipids as DGDG or MGDG, 0.02% phosphatidylglycerol. The sperm cells plus extender (shown in FIG. 4B) were frozen according to standard procedures, thawed for >45 seconds at 37° C. then co-stained with merocyanine 540 and Yo-Pro and analyzed on a ZE-5 flow cytometer using a 577/15 filter for emission and excitation using 488 laser.

Merocyanine 540 (M540) is a lipophilic dye that incorporates into membranes differently depending on the phase of the lipids and can be indicative of rippled gel, liquid-crystalline or perhaps even non-bilayer isotropic, phases. It incorporates into the hydrocarbon region of the bilayer but does not permeate the membrane. M540 binding may be indicative of diffusion potentials such as may be present when there is a monolayer associated with a bilayer. The binding of M540 causes a membrane perturbation based on its orientation. When such a perturbation is visualized, it can be indicative of a different membrane phase. The relative amplitude may be at 570 nm (amount of fluorescence).

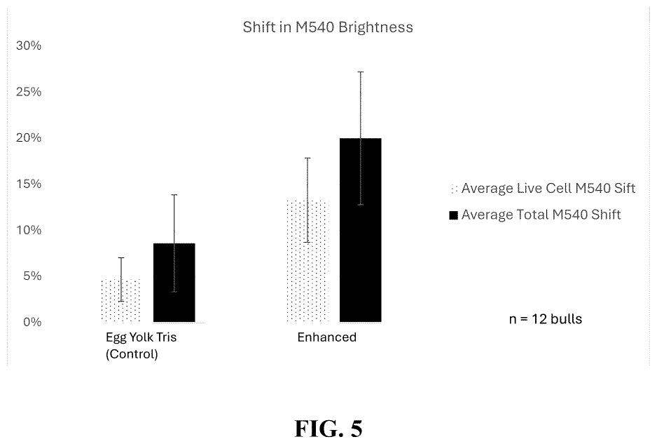

As can be seen in FIGS. 4A, 4B and 5, the M540 versus yo-pro displays a remarkably different profile in the enhanced versus the egg yolk tris buffer. The difference can be quantified by the total shift in stain intensity (or brightness).

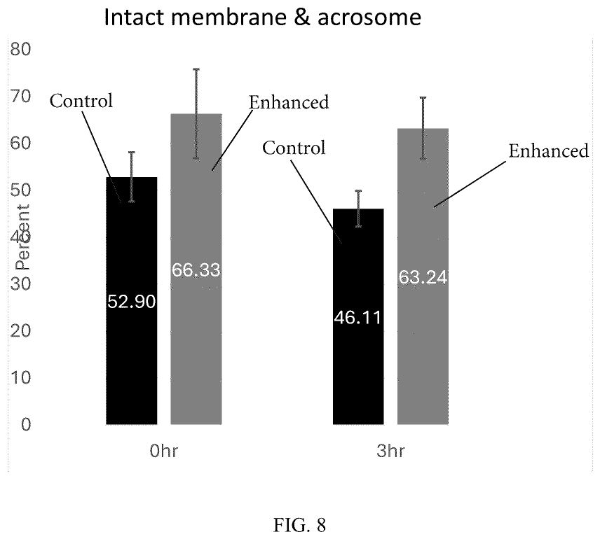

FIG. 8 shows a graph comparing the percentage of sperm that has an intact membrane and acrosome at 0 hours and 3 hours having been after thawed from cryopreservation. Sperm from a single ejaculate were cryopreserved in either a control group medium or an enhanced group medium with exogeneous lipids added. The thawed sperm were held at 37° C. The enhanced group medium had a greater percentage of intact sperm without cryopreservation damage at both times (P<0.05).

It is noted that in the past traditional membrane lipids may have been used with biological cells but they did not form a different structure such as a protective layer, a non-bilayer, or the like structure. Lipids that have a high proportion of cis-double bonds may be used. In embodiments, plant cell membrane components, phospholipids, sphingolipids, alone or in combination may be used. In some embodiments, exogeneous lipids may not include certain components such as but not limited to phosphatidylethanolamine, cholesterol, or the like.

As can be easily understood from the foregoing, the basic concepts of the present invention may be embodied in a variety of ways. It involves both cell preserving techniques as well as devices to accomplish the appropriate cell preservation. In this application, the cell preserving techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “collection” should be understood to encompass disclosure of the act of “collecting”—whether explicitly discussed or not—and, conversely, were there effectively disclosure of the act of “collecting,” such a disclosure should be understood to encompass disclosure of a “collection” and even a “means for collecting.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the below list of references or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

Thus, the applicant(s) should be understood to have support to claim and make a statement of invention to at least: i) each of the cell preservation devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. A method of processing biological cells comprising the steps of:
providing a biological cell;
adding exogeneous lipids to said biological cell before, during, or after in vitro processing of said biological cell;
forming a protective layer around at least part of said biological cell with said exogeneous lipids to create a stabilized biological cell;
in vitro processing said stabilized biological cell; and
substantially preserving a plasma membrane of said biological cell during said in vitro processing.

2. The method of claim 1 wherein said step of forming said protective layer around at least part of said biological cell comprises a step of forming said protective layer over at least part of said plasma membrane said biological cell.

3. The method of claim 1 wherein said step of forming said protective layer comprises a step of allowing said exogenous lipids to interact with said plasma membrane.

4. The method of claim 1 wherein said step of forming a protective layer around at least part of said biological cell with said exogeneous lipids to create said stabilized biological cell comprises a step of creating a membrane-stabilized biological cell.

5. A method of processing biological cells comprising the steps of:
providing a biological cell;
adding exogeneous lipids to said biological cell;
forming a protective layer around at least part of said biological cell with said exogeneous lipids;
in vitro processing said biological cell; and
substantially preserving a polarized state of said biological cell during in vitro processing with said protective layer.

6. The method of claim 5 wherein said step of substantially preserving said polarized state of said biological cell comprises a step of substantially preserving an electrical membrane potential of said biological cell during said in vitro processing.

7. The method of claim 5 wherein said step of substantially preserving said polarized state of said biological cell comprises a step of substantially maintaining a zinc-ion efflux ability of a sperm cell during said in vivo processing.

8. The method of claim 5 wherein said step of substantially preserving said polarized state of said biological cell comprises a step of substantially maintaining a zinc signature state of a sperm cell during said in vivo processing.

9. The method of claim 5 and further comprising a step of preserving said polarized state of said biological cell through in vitro processing of said biological cell until said polarized state of said biological cell no longer needs to be preserved.

10. The method of claim 9 wherein said step of said biological cell no longer needs to be preserved comprises a step of chosen from when said biological cell is at an appropriate place in uterine tract, when said biological cell has been used for in vitro fertilization, and when said biological cell has been used for in vivo fertilization.

11. The method of claim 5 and further comprising a step of substantially preserving a plasma membrane of said biological cell during said in vitro processing.

12. The method of claim 5 wherein said step of forming said protective layer around at least part of said biological cell comprises a step of forming over at least part of said plasma membrane said biological cell.

13. The method of claim 12 wherein said step of forming said protective layer around at least part of said biological cell comprises a step of allowing said exogenous lipids to interact with said plasma membrane

14. A method of processing biological cells comprising the steps of:
providing a biological cell having an intrinsic curvature of its plasma membrane;
adding exogeneous lipids to said biological cell before, during, or after in vitro processing of said biological cell;
forming a protective layer mimicking said intrinsic curvature around at least part of said plasma membrane with said exogeneous lipids to create a membrane-stabilized biological cell;
in vitro processing said membrane-stabilized biological cell; and
substantially preserving said plasma membrane of said biological cell during said in vitro processing.

15. The method of claim 14 wherein said step of forming said protective layer around at least part of said plasma membrane comprises a step of forming an isotropic, non-lamellar protective layer over at least part of said plasma membrane.

16. The method of claim 14 wherein said step of substantially preserving said plasma membrane comprises the step of inhibiting substantial change of an intrinsically functioning plasma membrane during in vitro processing of said biological cell.

17. The method of claim 14 wherein said step of forming said protective layer comprises a step of allowing said exogenous lipids to interact with said plasma membrane.

18. A method of restoring damaged cell membranes comprising the steps of:
providing a damaged plasma membrane of a biological cell;
adding exogenous lipids to said damaged plasma membrane of said biological cell;
forming a protective layer over at least part of said damaged plasma membrane of said biological cell with said exogenous lipids;
allowing said exogenous lipids to interact with said damaged plasma membrane of said biological cell; and
repairing said damaged plasma membrane from said interactions of said exogenous lipids with said damaged plasma membrane.

19. The method of claim 18 wherein said step of allowing said exogenous lipids to interact with said damaged plasma membrane of said biological cell comprises a step of integrating said exogenous lipids with endogenic lipids of said damaged plasma membrane.

20. The method of claim 18 wherein said step of providing said damaged plasma membrane of a biological cell comprises a step of providing a damaged plasma membrane of a biological cell that was damaged from in vitro processing of the biological cell.

21-49. (canceled)
